SEQUENCE LISTING (11 sequences)

SEQ ID NO: 1 - protein, 86 aa, Human immunodeficiency virus type 1
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 2 - DNA, 310 bp, Human immunodeficiency virus type 1
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaa

SEQ ID NO: 3 - DNA, 28 bp, Artificial
  PCR primer; PBAMU
cgcggatccatggagccagtagatccta

SEQ ID NO: 4 - DNA, 36 bp, Artificial
  PCR primer; R8
gttatgaaacaaacttgggaatgaaaggaagacttt

SEQ ID NO: 5 - DNA, 28 bp, Artificial
  PCR primer; PHINDR
ccccaagcttcactaatcgaatggatct

SEQ ID NO: 6 - DNA, 36 bp, Artificial
  PCR primer; U8
aaagtcttcctttcattcccaagtttgtttcataac

SEQ ID NO: 7 - DNA, 42 bp, Artificial
  PCR primer; R9
aaaggaagactttttagaataggaattggtagaagcagtttt

SEQ ID NO: 8 - DNA, 42 bp, Artificial
  PCR primer; U10
taaaaagtcttcctttcattcccaagtttctttcataacaaa

SEQ ID NO: 9 - DNA, 41 bp, Artificial
  PCR primer; R11
gaaagaaacttgggaatgaaaggaagactttttagaatagg

SEQ ID NO: 10 - DNA, 5315 bp, Artificial
  Plasmid pM1800
tggcgaatgccttaattaaggcggggcacaactcaatttgcgggtactgattaccgcagcaaagaccttaccccgaaaaaatccaggctgctggctgacacgatttctgcggtttatctcgatggctacgagggcagacagtaagtggatttaccataatcccttaattgtacgcaccgctaaaacgcgttcagcgcgatcacggcagcagacaggtaaaaatggcaacaaaccacccgaaaaactgccgcgatcgcgcctgataaattttaaccgtatgaatacctatgcaaccagagggtacaggccacattacccccacttaatccactgaagctgccatttttcatggtttcaccatcccagcgaagggccatccagcgtgcgttcctgtatttccgactggcgcgccattcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 11 - DNA, 4914 bp, Artificial
  Plasmid pET8cTat
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagttattgctcagcggtggcagcagccaactcagcttcctttcgggctttgttagcagccggatccgttcactaatcgaatggatctgtctctgtctctctctccaccttcttcttctattccttcgggcctgtcgggtcccctcgggattgggaggtgggttgctttgatagagaaacttgatgagtctgactgccttgaggaggtcttcgtcgctgtctccgcttcttcctgccataggagatgcctaaggcttttgttatgaaacaaacttggcaatgaaagcaacactttttacaatagcaattggtacaagcagttttaggctgacttcctggatgcttccagggctctagtctaggatctactggctccatggtatatctccttcttaaagttaaacaaaattatttctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

(19) United States
(12) Patent Application Publication
(10) Pub. No.: US 2004/0005330 A1
(43) Pub. Date: Jan. 8, 2004

(54) Mutated HIV Tat

(76) Inventors: Jay Rappaport, Bala Cynwyd, PA (US); Michel Klein, La Tour de Salvagny (FR); Jean Francois Zagury, Paris (FR)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/310,740

(22) Filed: Dec. 5, 2002

Related U.S. Application Data
(60) Provisional application No. 60/339,607, filed on Dec. 11, 2001.

(51) Int. Cl. A61K 39/21; C07H 21/04; C07K 14/16; C12P 21/02; C12N 5/06; C12N 15/867

(52) U.S. Cl. 424/188.1; 530/350; 435/69.3; 435/320.1; 435/325; 536/23.72

(57) ABSTRACT

The present invention provides a Tat protein wherein all the cysteine residues of the cysteine-rich domain have been replaced with another amino acid, preferably with serine, nucleic acids encoding it, and methods of using it to elicit a humoral and cellular immune responses in a mammal. The Tat protein of the invention is therefore useful, inter alia, for prophylactic and/or therapeutic anti-HIV use as well as raising anti-native Tat antibodies in mammals.

### BACKGROUND OF THE INVENTION

This application claims priority to U.S. Provisional Application Serial No. 60/339,607, filed Dec. 11, 2001.

### FIELD OF THE INVENTION

This invention relates to the field of modified HIV Tat nucleic acids and proteins as well as its combination with early HIV proteins and their use in studying the biological mechanism of HIV infection and in vaccine compositions for prophylaxis and treatment of HIV/AIDS.

### SUMMARY OF THE RELATED ART

HIV Tat protein is an essential viral protein for HIV pathogenesis. It transactivates HIV gene expression by binding to the Trans Activation Response (TAR) element of the HIV RNA Long Terminal Repeat (LTR) region. Tat is released by infected cells in which it is expressed (soluble Tat or sTat) and taken up by other HIV infected cells, where it can enter the nucleus and transactivate HIV gene expression. Extracellular Tat induces expression of HIV co-receptors on target cells, thereby further promoting virus spreading. See generally Noonan et al., Advances in Pharmacology 48, 229 (2000).

Tat also plays a role in HIV-induced immunosuppression. Id. For example, Cohen et al., Proc. Natl. Acad. Sci. USA 96, 10842 (1999), reported that Tat is strongly immunosuppressive, both immediately after immunization of mice with sTat and in seroconverting humans. Tat has also been linked to induction of T-cell anergy and T-cell apoptosis, Ross, Leukemia, 15, 332 (2001). Furthermore, Tosi et al., Eur. J. Immunol. 30, 19 (2000), demonstrated that a modified HIV-1 Tat can act as a immunosuppressor by inhibiting HLA class II expression necessary for triggering both cellular and humoral responses against pathogens.

The Tat protein is an 86-102 (depending on the HIV strain) amino acid protein encoded by two exons. The first, highly conserved exon contains four functional domains, including the amino-terminal domain (amino acids 1-21), the cysteine-rich domain (amino acids 22-37), the core domain (amino acids 38-48), and the basic domain (amino acids 49-57), which is essential for cellular uptake. The cysteine-rich domain is highly conserved and has been reported as being important for the Tat transactivating activity. Individual mutation in six of the seven cysteines eliminates Tat function. Jeang in HIV-I Tat: Structure & Function, pp. 3-18, Los Alamos National Laboratory (Ed.) Human Retroviruses & AIDS Compendium III.

Because of its essential role in HIV expression and propagation, Tat has been suggested and studied as a possible vaccine. Goldstein, Nature Medicine, 1, 960 (1996). Cafaro et al., Nature Medicine 5, 643 (1999) reported that vaccination of cynomolgus monkeys with a biologically active HIV-1 Tat protein is safe, elicits a broad (humoral and cellular) specific immune response and reduces infection of the highly pathogenic simian-human immunodeficiency virus (SHIV)-89.6P to undetectable levels.

For human use suppression or inactivation of Tat activity has been suggested as a route for prophylaxis and/or treatment of HIV infection. E.g., Goldstein, WO 95/31999. Tat protein that has been modified to reduce or eliminate its transactivating activity while maintaining its immunogenicity has been proposed.

Cohen et al. (supra) reported that oxidation of Tat preserves immunogenicity of the protein while inactivating Tat's immunosuppressive effects.

Le Buanec and Bizzini., Biomed & Parmacother. 54, 41 (2000), reported on chemical inactivation of Tat, e.g., formaldehyde, glutaraldehyde, and dithionitrobenzoate treatment as well as amidination of lysyl residues, modification of arginyl residues, blockade of sulfhydryl groups by dithionitrobenzoate treatment, maleimidation, carboxymethylation, and carboxyamidation. They found that such chemical modification resulted in a Tat protein with a partial or complete loss of biological activity but retention of partial to complete antigenicity and immunogenicity in mice compared to native Tat. Zagury et al. (U.S. Pat. No. 6,200,575) also discloses formaldehyde and glutaraldehyde inactivation of Tat.

Another approach has been modification of the protein by mutation. Caselli et al. investigated two tat genes mutated in the transactivating domain for their ability to elicit an immune response to wild-type Tat in a mouse model. The polypeptides encoded by the two genes, tat₂₂ (Cys²²→Gly) and tat_{22/37} (Cys²²→Gly and Cys³⁷→Ser), lack HIV transactivating activity and block wild-type Tat. Caselli et al. injected mice with DNA plasmids containing the tat₂₂ and tat_{22/37} genes and tested for humoral and cellular response to wild-type Tat. A humoral response suggestive of a Th1 profile was detected after the third immunization, and mean titers and the number of responder mice increased following three additional boosts and treatment with bupivacaine (which facilitates DNA uptake in muscle cells and enhances DNA immunization). The response was comparable to DNA immunization with the wild-type tat gene.

Caselli et al. also immunized mice with wild-type Tat protein and observed both humoral and cellular responses. Antibody titers were higher in the Tat-immunized mice compared to the tat₂₂ and tat_{22/37} immunized mice, although epitope reactivities were more restricted and a Th-2-like response observed. The authors speculated that the differences in DNA and protein immunization response were likely due to protein sensitivities to air, light, and temperature and differences in presentation of the two to the immune system. Caselli et al. asserted that DNA immunization seemed preferable due to the presence of a cellular response characteristic of a Th1 reaction.

Zagury, (U.S. Pat. No. 6,200,575) discloses the use of inactivated Tat and various forms of inactivated Tat as immunogens for prophylactic or therapeutic immunizations to fight HIV disease.

Tosi et al., (supra) reported on a tat_{22/37} (Cys²²→Gly and Cys³⁷→Ser) and a tat₃₇ (Cys³⁷→Ser) mutant, both transfected into T and monocytic cell lines. Both mutants were re-ported to strongly down-modulate constitutive as well as IFN-γ-inducible HLA class II gene expression in vitro, suggesting that these mutants retain the immunosuppressive function of the native polypeptide.

Goldstein, Nature Medicine, supra, suggested that a consensus sequences of 21 known HIV-1 Tat proteins could be used as the immunogen in a vaccine and further suggested Cys→Ser substitutions could be made at positions 22, 25, 27, and/or 37 to block transactivation without affecting the immunogenic domains.

Goldstein WO 95/31999 suggested inactivation of Tat by deletion at the amino or carboxy terminus or deletion or replacement of native cysteine residues to interfere with naturally-occurring disulfide bonds.

Loret (WO 00/61067) discloses Tat protein mutated in the cysteine rich region. Most particularly, Loret specifically considers Tat OYI, which corresponds to a Tat protein having a natural Cys²²→Ser mutation.

Furthermore, Osterhaus et al. has demonstrated the presence of Tat and Rev-specific CTL in seropositive long term non progressors whereas these CTLs were not found in patients progressing to disease. In addition immunization of macaques with a combination of vectors expressing the SIV tat and rev genes protected the animals against pathogenic SIV challenge. Vaccine 17, 27-31, 1999; U.S. Pat. No. 6,024,965.

A recent study by Addo et al. (Proc. Natl. Acad. Sci. USA vol. 98, 1781-1786) demonstrated that controllers (HIV-1 infected individuals capable of controlling viremia without medication) had CTLs targeting more epitopes in Tat relative to individuals on drug treatment. Furthermore, the anti-Tat CTL responses were also of higher magnitude in controllers.

More recently, Allen et al. demonstrated that Tat-specific CTLs are involved in controlling wild-type virus replication during SIV infection of rhesus macaques. Nature 407, Sept 2000, 386-390.

Despite the tremendous effort that has been dedicated to the study of HIV, Tat, early proteins and their role in AIDS, all of the molecular biological mechanisms of HIV in general and Tat in particular are not completely known or understood. A composition and method for HIV/AIDS prophylaxis and treatment has also remained elusive. Accordingly, there still remains a need for an HIV/AIDS vaccine as well as useful research tools to study HIV infection.

All patents and other publications recited herein are hereby incorporated by reference in there entirety.

### SUMMARY OF THE INVENTION

The present invention is based on the discovery that modification of HIV Tat protein in the cysteine rich domain by replacing all the cysteine residues with other amino acids, preferably serine, results in a modified Tat protein that retains its immunogenicity, is unable to transactivate HIV expression, is not immunosuppressive, and is able to induce neutralizing antibodies. The present invention comprises also the simultaneous use of tat, rev and nef genes to elicit broad HIV specific T cell responses (including CD4 and CD8 as well as innate immunity). This combination of features makes the modified Tat protein of the invention as well as its combination with early proteins useful both as a vaccine as well as a research tool to study the molecular and systemic mechanisms involved in HIV infection.

The present invention thus provides a Tat protein comprising a mutated cysteine-rich domain wherein all the cysteine residues of the cysteine-rich domain have been replaced independently with another amino acid.

According to a specific embodiment each cysteine residue of the cysteine-rich domain is a conservative substitution and is preferably a serine.

In another aspect, the invention relates to a nucleic acid encoding the Tat protein as defined above as well as an expression vector comprising said nucleic acid. In alternative embodiments, the said vector further comprises a DNA sequence encoding Nef and Rev proteins. According to a preferred embodiment, the DNA sequence encoding the Rev protein is inserted anywhere into the nef DNA sequence encoding amino acids 150-179 of the Nef protein.

In another aspect, the invention provides a composition comprising the above-defined Tat protein or expression vector in combination with a carrier and optionally an adjuvant, especially at least one Th1 adjuvant. Such composition is use for in vitro and in vivo administration both as an anti-HIV vaccine as well as for the purpose of studying HIV infection.

The present invention also relates to a method of eliciting a humoral and cellular immune response in a mammal comprising administering the above-defined composition to the mammal. According to a specific embodiment, the composition comprising the Tat protein of the invention is administered simultaneously or sequentially with the composition comprising the expression vector of the invention.

The foregoing merely summarizes certain aspects of the invention and is not intended, nor should it be constructed, as limiting the invention in any manner. Additional details of the invention are provided below. All patents, patent applications, and other publications recited in this specification are hereby incorporated by reference in their entirety.

### BRIEF DESCRIPTION OF THE DRAWINGS

FIG. 1 displays the results of immunosuppressive activity of various Tat measured in vitro by a lymphoproliferation assay.

FIG. 2 displays anti-Tat_{IIB} IgG ELISA titers of guinea pigs immunized with various Tat proteins.

FIG. 3 displays the results of the transactivation assay.

FIGS. 4A-B give the DNA sequence of plasmid pM1800 (SEQ ID NO.: 10).

FIG. 5 gives the plasmid map of pET8cTat.

FIGS. 6A-B give the DNA sequence of pET8cTat (SEQ ID NO.: 11).

### DETAILED DESCRIPTION OF THE INVENTION

In a first aspect, the invention thus provides a Tat protein comprising a mutated cysteine-rich domain wherein each cysteine residue of the cysteine-rich domain has been replaced with another amino acid, preferably a conservative amino acid, most preferably a serine.

As used herein a “Tat protein” means any naturally occurring Tat protein obtained from any HIV-1, HIV-2 or SIV strain, including laboratory and primary isolates. The Tat protein is obtained preferably from a HIV-1 strain and more particularly from a HIV-1 IIIB strain. Two kinds of Tat proteins have been disclosed in the literature i.e., Tat proteins having a short sequence of 86 amino acids and Tat proteins having a longer sequence of up to 99 to 102 amino acids. This difference in size has been attributed to the variable length of the second exon encoding the protein. These two types of proteins fall under the scope of the invention. The amino acid sequences of a large number of Tat proteins are known and available, e.g., “Human Retroviruses and AIDS 1999: A Compilation and Analysis of Nucleic Acid and Amino Acid Sequences,” Kuiken et al., Eds., Theoretical Biology and Biophysics Group, Los Alamos National Laboratory, Los Alamos, N. Mex., and http://hiv-web.lanl.gov/, and any of these can be used in the present invention. The Tat protein is composed of various conserved functional domains, and comprises particularly a highly conserved cysteine-rich domain. This definition also encompasses the said Tat proteins in which mutations have been introduced with the proviso that the said proteins contain a mutated cysteine-rich domain as defined below and remain devoid of any transactivating and immunosuppressive activity and further remain capable of inducing neutralizing antibodies and a cellular immune response. The Tat protein of the invention is preferably Tat IIIB and corresponds most preferably to Seq ID No. 1.

As used herein, the “mutated cysteine-rich domain” is the sequence corresponding to amino acids 22 to 37 of the Tat protein wherein each cysteine residue at positions 22, 25, 27, 30, 31, 34 and 37 have been independently replaced with another amino acid, corresponding preferably to a conservative substitution and most preferably to a serine residue. This definition intends also to include cysteine-rich domains in which in addition to the above-mentioned mutations, additional conservative substitution(s) have been introduced in positions different from positions 22, 25, 27, 30, 31, 34 and 37. Taking as a reference the cysteine-rich domain of Tat IIIB, this definition includes all cysteine-domains having a similarity with IIIB cysteine-rich domain of at least 50%, preferably of at least 75%, most preferably of 100%.

A “conservative amino acid substitution” is a substitution of a native amino acid residue with a normative residue such that there is little or no effect on the polarity or charge of the amino acid residue at that position. A “conservative amino acid substitution” also encompasses non-naturally occurring amino acid residues that are typically incorporated by chemical peptide synthesis rather than by synthesis in biological systems. These include peptidomimetics, and other reversed or inverted forms of amino acid moieties.

Naturally occurring residues may be divided into classes based on common side chain properties:

1) hydrophobic: norleucine, Met, Ala, Val, Leu, Ile;

2) neutral hydrophilic: Cys, Ser, Thr;

3) acidic: Asp, Glu;

4) basic: Asn, Gln, His, Lys, Arg;

5) residues that influence chain orientation: Gly, Pro; and

6) aromatic: Trp, Tyr, Phe.

For example, non-conservative substitutions may involve the exchange of a member of one of these classes for a member from another class.

In making such changes, the hydropathic index of amino acids may be considered. Each amino acid has been assigned a hydropathic index on the basis of its hydrophobicity and charge characteristics. The hydropathic indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al., 1982, J. Mol. Biol. 157:105-31). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within +1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within ±2 is preferred, those which are within +1 are particularly preferred, and those within ±0.5 are even more particularly preferred. One may also identify epitopes from primary amino acid sequences on the basis of hydrophilicity. These regions are also referred to as “epitopic core regions.”

Desired amino acid substitutions (whether conservative or non-conservative) can be determined by those skilled in the art at the time such substitutions are desired.

The term “similarity” refers to a measure of relatedness that includes both identical matches and conservative substitution matches between two sequences as determined by a particular mathematical model or computer program (i.e., “algorithms”) by inserting gaps, if required, in one or both sequences. A suitable programs available for public use is FASTA. If two polypeptide sequences have 10 of 20 identical amino acids, for example, and the remainder are all non-conservative substitutions, then the percent identity and similarity would both be 50%. If in the same example there are five positions in which there are conservative substitutions (in addition to the 10 identical residues), then the percent identity remains 50%, but the percent similarity would be 75% (15/20).

In a preferred embodiment of the Tat protein of the invention, amino acid residues at positions 22, 25, 27, 30, 31, 34, and 37 are serine residues (herein called Tat7C/S). According to a preferred embodiment, Tat7C/S corresponds to Tat IIIB 7C/S.

In another embodiment, the Tat protein of the present invention is further modified by chemically methods such as those disclosed in U.S. Pat. No. 6,200,575.

Amino acid numbering used herein is based on the sequence of the HIV-1 viral strain III B. The Tat protein of this strain is (SEQ. ID. NO.: 1): MEPVDPRLEPWKHPGSQPKTACTNCYCKKCCFHCQVCFITKALGISYGRKKRRQRRRPPQGSQTHQVSLSK QPTSQSRGDPTGPKE

Whenever a number of an amino acid residue or sequence is used in reference to a sequence other than from the IIIB strain, that number refers to the residue or sequence that corresponds to the numbered residue or sequence in the IIIB Tat.

The Tat proteins of the invention can be made routinely using methods known in the art. The proteins can be synthesized or, preferably, expressed from a vector in a suitable expression system. Vectors and expression of the encoded Tat protein of the invention is described fully below. When the Tat protein is produced by chemical synthesis, it is possible either to produce it in the form of one sequence or in the form of several sequences that are subsequently linked together in the correct order. The chemical synthesis may be carried out on solid phase or in solution, these two technologies being well known to the person skilled in the art and are described for example by the following authors: Atherton and Shepart “solid phase peptide synthesis” (IRL press Oxford, 1989); Houbenweyl “Method der organischen chemie” editor E. Wunsch vol 15-I and II, Stuttgart 1974; Dawon PE and al “Synthesis of proteins by native chemical ligation” Science, 1994, 266 (5186): 776-9; Kochendoerfer GG and al “Chemical protein synthesis” Curr. Opin. Chem. Biol., 1999, 3(6):665-71; and Dawson PE and al “Synthesis of native proteins by chemical ligation” Annu. Rev. Biochem. 2000, 69: 923-60. The protein thus produced may be easily isolated and purified by methods well known in the art.

The protein of the invention may also be produced by recombinant technologies well known in the art. These methods are described in details in the last edition of “Molecular Cloning: A Molecular Manual” by Sambrook et al., Cold Spring Harbor, supra. In such a case, the DNA sequence encoding the Tat protein of the invention is first produced by directed mutagenesis starting from the wild-type DNA sequence encoding Tat. Such a step may be carried out by PCR using primers containing the DNA sequence encoding the mutation(s) to be introduced. The mutated DNA sequence is then inserted into an appropriate expression vector. The thus obtained recombinant vector is then used to transform appropriate host cells to express the mutated Tat protein. The protein thus produced is isolated and purified using methods well known in the art. A process of expression and purification of the protein according to the invention is described in details in the attached examples. The process of the invention leads advantageously to a highly purified monomeric Tat protein which does not form any aggregates.

Concerning the “expression vector,” any expression vector classically used for the expression of recombinant proteins can be used to produce the Tat protein of the invention. “Expression vectors” thus encompass live expression vectors such as viruses and bacteria as well as plasmids. Vectors in which the expression of the Tat DNA sequence is controlled by an inducible or non-inducible strong promoter are advantageously used. Expression vectors may include a selection marker such as, for example, an antibiotic resistance gene (such as Kanamycin) or dihydrofolate reductase gene.

Non-limitative examples of expression vectors that can be used in the process of production of the Tat protein of the invention include: pET28 (Novagen), pBAD (Invitrogen) plasmids; viral vectors such as baculovirus, adenovirus, adeno-associated virus (MV), poxvirus (including avian pox, fowl pox, and preferably the attenuated vaccinia vector NYVAC (U.S. Pat. No. 5,364,773) or MVA (modified vaccinia virus Ankara, Swiss Patent No. 568,392 and U.S. Pat. No. 5,185,146), and the attenuated canarypox vector ALVAC (U.S. Pat. Nos. 5,756,103; 5,990,091), poliovirus, alphavirus, VSV, herpes and retroviral vectors, as well as bacterial vectors such as salmonella, shigella and BCG.

To obtain the expression of the Tat protein, any host cell classically used in association with the above-mentioned vectors can be used in the present invention. Non limitative examples of such host cells include cells from E. coli such as BL21(λDE3), HB101, Topp 10, CAG 1139, cells from bacillus, and eukaryotic cells such as Vero, BHK, MRC5, MDCK, PERC-6, and CHO cells.

The expression system preferably used in the present invention corresponds to the pM 1815/E. coli cells.

In another aspect, the invention relates to the nucleic acid sequences encoding the above-defined Tat protein of the invention. The nucleotide sequences of a large number of tat genes are known and available, e.g., on the web site: http://hiv-web.lanl.gov/. Nucleic acid numbering used herein is based on the following tat DNA sequence from HIV-1 viral strain III B (Seq. ID. No. 2):

atggagccag tagatcctag actagagccc tggaagcatc
caggaagtca gcctaaaact gcttgtacca attgctattg
taaaaagtgt tgctttcatt gccaagtttg tttcataaca
aaagccttag gcatctccta tggcaggaag aagcggagac
agcgacgaag acctcctcaa ggcagtcaga ctcatcaagt
ttctctatca aagcaaccca cctcccaatc ccgaggggac
ccgacaggcc cgaaggaata gaagaagaag gtggagagag
agacagagac agatccattc gattagtgaa

The bold/underline codon indicates a stop codon at position 259 (with an X in the corresponding position in the amino acid sequence) in the IIIB Tat, which, accordingly is 86 amino acids long. A number of naturally occurring Tat sequences have a Glu or Ser codon in place of this stop codon and have an additional 14 or more amino acid residues at the carboxy terminal end.

Whenever a number of a nucleic acid residue is used in reference to a sequence other from the IIIB strain, that number refers to the residue that corresponds to the numbered residue in the Seq ID No. 2 sequence.

When a first nucleic or amino acid residue or sequence within a first polynucleotide or polypeptide (respectively) aligns with a second nucleic or amino acid residue or sequence within a second polynucleotide or polypeptide (respectively) when the two polynucleotides or polypeptides are brought into alignment using any art recognized alignment algorithm, e.g., SIM (Xiaoquin et al., Advances in Applied Mathematics 12, 337 (1991)), the first nucleic or amino acid residue or sequence within a first polynucleotide or polypeptide (respectively) are said to “correspond” one to the other.

The codons of the nucleic acids of the invention can be advantageously optimized to improve the expression level, the selection of the optimized codons depending on the selected host cells.

In a third aspect, the invention comprises an expression vector encoding the nucleic acid of the second aspect of the invention. Expression vectors into which the nucleic acids of the second aspect of the invention may be inserted are well known in the art and can be routinely selected by those of ordinary skill in the art based primarily on the host system into which the vector is to be inserted. Methods for inserting the nucleic acids of the second aspect of the invention into vectors are well known and routinely applied. E.g., Sambrook et al., “Molecular Cloning: A Laboratory Manual” vols. 1-3 (3^{rd} Edition, Cold Spring Harbor Laboratory Press, Cold Spring Harbor, N.Y. 2001).

Expression vectors that can be employed in this aspect of the invention have been described in detail in the section regarding the process of production of the Tat protein. The expression vectors of the present invention can be used either for the production of the Tat protein or directly as an active vaccine component of a composition of the invention. When the expression vector is used as a vaccine component, the expression vector to be used does not comprise any selection marker and corresponds to a viral vector such as adenovirus, poxvirus (including fowl pox, avian pox, and preferably the attenuated vaccinia vector NYVAC (U.S. Pat. No. 5,364,773) or MVA (modified vaccinia virus Ankara, Swiss Patent No. 568,392 and U.S. Pat. No. 5,185,146), and the attenuated canarypox vector ALVAC (U.S. Pat. Nos. 5,756,103; 5,990,091), poliovirus, alphavirus, VSV, herpes retroviral vector, or a bacterial vector such as salmonella, shigella or BCG, or a plasmid DNA vectors including layer DNA vectors.

In one embodiment of this aspect of the invention, the nucleic acid encoding the modified Tat polypeptide of the invention is the only HIV/SIV immunogen encoded by the vector.

In a preferred embodiment, the vector according to this aspect of the invention further comprises nucleic acid sequences encoding the Rev and Nef HIV-1 proteins. Numerous wild-type rev and nef nucleic acid sequences are known. FIGS. 9-11 and 15-17 display many of them, and we contemplate that any of those displayed as well as consensus sequences of any two or more of these sequences can be used in the invention. In this embodiment, the vector of the invention comprises a nucleic acid sequence according to the second aspect of the invention and both a rev and nef sequence, and the vector express the mutated Tat protein of the invention and Rev and Nef proteins in the intended host. Preferably, in this embodiment the rev DNA sequence is inserted into the nef DNA sequence. Preferably, the rev DNA sequence is inserted anywhere into the region coding for amino acids 150-179 of Nef, thus producing an inactivated Nef protein without altering the CTL epitopes of the protein.

As used here “Nef and Rev proteins” means any naturally occurring Rev and Nef proteins obtained form any HIV-1, HIV-2 or SIV strain including laboratory and primary isolates. The Rev and Nef proteins are obtained preferably from a HIV-1 strain. The DNA sequences inserted in the expression vector of the invention comprises preferably the consensus sequences of the DNA sequences encoding Rev and Nef or DNA fragments thereof coding for CTL epitopes. The amino acid and nucleotide sequences of Rev and Nef as well as the CTL epitopes thereof so far identified can be download from the web site: http://hiv-web.lanl.gov/.

In a preferred embodiment, the expression vector expresses the DNA sequence encoding the Tat protein under one promoter and the DNA sequences encoding Rev and Nef under another promoter. This construct advantageously produces an immunologically active Tat protein capable of being secreted by mammalian cells, taken up by mammalian cells, is presented as antigen and is recognized by immune cells and/or specific antibody.

The modified Tat proteins of the invention have several uses. They can be used alone or as a component of a prophylactic or therapeutic vaccine, where its inability to transactivate HIV gene expression and induce immunosuppression in a host while retaining its immunogenicity and capacity to produce neutralizing antibodies and cellular immune response make it both safe and effective for HIV infection prophylaxis and treatment.

The transactivating and immunosuppressive activities of the Tat protein can be easily determined by the CAT assay and the immunosuppression assay, respectively, as described in the attached examples. The induction of neutralizing antibodies can be easily demonstrated by the neutralization assay as described in the attached examples.

The present invention thus also provides compositions, especially vaccines, comprising a Tat protein and/or an expression vector as defined above in combination with a suitable carrier.

The nature of the carrier will vary depending on the intended application. For example, for in vitro assays, the carrier can be a simple buffer solution. For prophylactic or therapeutic purposes, the carrier can be any pharmaceutically acceptable carrier, many of which are known in the art. A pharmaceutically acceptable carrier will also be desirable for uses in vivo other than treatment or prophylaxis, e.g., raising anti-Tat antibodies for use in assays or treatment.

Methods of making pharmaceutical compositions are well known and can be routinely used to make pharmaceutical compositions according to the fourth and fifth aspects of the invention. E.g., “Remington: The Science and Practice of Pharmacy,” by Alfonso R. Gennaro (20th edition, Lippincott, Williams & Wilkins, Philadelphia, Pa., 2000).

According to one embodiment, the composition comprises Tat7C/S in combination with a pharmaceutically acceptable carrier. Such a composition may be stored in lyophilized form and reconstituted in an injectable solution before injection.

The composition of the invention may include one adjuvant such as a Th1 adjuvant (e.g., CpG sequences or MPL and MPL analogs), or a Th2 adjuvant (e.g., alum, emulsions, minerals) or a combination adjuvant including at least one Th1 adjuvant.

As part of a vaccine the Tat protein of the invention can also be used in a lipidated form comprising a lipidic part covalently linked to the Tat protein. Lipidic parts appropriate to form such lipidated Tat as well as a process of preparation of the same can be found e.g., in U.S. Pat. No. 5,993,823. The lipidated Tat protein comprises preferably a N-ε-lysylpalmytoyl residue linked at the COOH terminal function of the Tat protein.

As part of a vaccine, it can be the sole immunogen or one of several. The Tat protein can be used as the sole immunogen of therapeutic anti-HIV vaccine. Preferably the protein of the invention is used in combination with an expression vector expressing the Tat protein of the invention in combination with Rev and Nef in order to produce an anti-HIV prophylactic or therapeutic vaccine. Tat, Rev and Nef are HIV proteins expressed early during the infection cycle, before production of infectious virions. These proteins are processed and CTL epitopes are expressed in the context of HLA class I antigen on the surface of HIV-infected cells. The advantage of immunizing humans against these three proteins altogether is to induce cytotoxic T cells capable of killing HIV infected cells before virions can be produced thus eradicating infected cells and preventing HIV replication and spreading. Also, one of the functions of the viral Nef protein is to down-regulate MHC class-1 molecule expression on the cell surface and thereby confer resistance to immune recognition by CD8 cells. Once the structural proteins are made, there is presumably sufficient Nef already present to confer resistance to cytotoxic T cells. The Nef used in this embodiment as a vaccine, therefore, should be devoid of this activity.

Furthermore, the protein and the expression vector of the invention may also be combined with other subunits HIV immunogens or vectors encoding the same such as Env, Gag, Pol, Vpr, Vpu and Vif. Advantageously, the Tat protein of the invention may be combined with the AL-VAC constructions, especially ALVAC 1452 and 1433 as disclosed in U.S. Pat. No. 5,990,091.

Such vaccines can be prepared by standard methods well known to those of ordinary skill in the art with standard vaccine pharmaceutical carriers and, preferably, with an adjuvant.

In a sixth aspect, the invention provides a method of eliciting a humoral and cellular immune response in a mammal, comprising administering to a subject (preferably human) one or more compositions according to the fourth and/or fifth aspect of the invention to elicit humoral and cellular immune responses.

“Cellular immune response” means induction of a specific CD4 T cell response optionally in association with a specific CD8 T cell response and an innate immune response.

CD4 T cell responses can be monitored upon in vitro recall of peripheral or splenic mononuclear cells with the antigen used to immunized animals. Lymphoproliferative responses as well as cytokine inductions (Th1/Th2 balance) can be measured (for a review see MK Jenkins, Annu rev Immunol. 2001, 19, 23-45).

CD8 T cell responses can be evaluated (ex vivo or upon re-stimulation of mononuclear cells) either using 1) a standard Chromium release assay which directly measures antigen specific lytic activity (P. Brossard et al., Blood, 90, 1594-1599) or using IFNγELISPOT or ICC (intracellular cytokine) assays that both measure the ability of CD8 cells to be stimulated by a 9 mer peptide specific for the antigen versus an irrelevant 9 mer peptide (Carvalho L H et al., J. Immunol. Methods 2001; 252, 207-18) for IFNγELISPOT and (C King et al., Nature Medicine, 7, 206-212) for ICC.

Innate immune responses can be monitored by measuring the leves of pro-inflammatory (IL-6, TNFα) and/or anti-viral (type I interferons) cytokines in the serum of immunized animals or upon in vitro antigen specific re-stimulations. The early stimulation of innate immunity can also be evaluated by assessing the ex vivo activation status of antigen presenting cells (monocytes, dendritic cells) and NK cells that are derived from recently immunized animals (L Krishnan et al., J. Immunol. 2001, 166, 1885-1893).

According to a preferred embodiment, a composition of the invention comprising a Tat protein is administered simultaneously or sequentially, preferably co-administered, with a composition comprising an expression vector of the invention, preferably an expression vector expressing in addition to the Tat protein of the invention the Rev and Nef proteins.

Suitable amounts of protein for vaccine and other in vivo applications are 10 to 500, preferably 20 to 200 μg per dose. Suitable amounts of viral expression vectors are in the range of 10⁴ to 10¹¹ pfu, and suitable amounts of plasmid expression vectors is 0.1 to 5 mg per dose.

Administration according to this aspect of the invention can thus be of a protein composition according to the fourth aspect of the invention, a vector according to the fifth aspect of the invention, or both, either simultaneously or sequentially. Furthermore, administration may comprise compositions of more than one protein, or expression vector. For example, one or a combination of composition comprising DNA plasmid plus a viral vector or two vectors expressing the same genes can be administered (e.g., DNA plasmid-tat/rev/nef+Pox-tat/rev/nef or Al-phavirus-tat/rev/nef+Pox-tat/rev/nef or). In an alternative embodiment, administration according to this aspect of the invention can be a combination of vectors carrying different genes (e.g., vector-tat/rev/nef+vector-gag/pol/env). In each instance, the number of injections is preferably 2 to 5 for each vector. Furthermore, the number of injections is also preferably of 2 to 5 for the composition comprising the Tat protein of the invention.

Administration of the composition of the invention can be carried out by intradermal, mucosal route or preferably by intramuscular injection.

The method of this aspect of the invention is useful for prophylactic and therapeutic treatment of HIV infection. The method is also useful to raise anti-Tat antibodies in a healthy mammal or a mammal infected with HIV without further harming the mammal. The antibodies thereby raised can be harvested and used for treatment, for assays, and for the study of the molecular and systemic effects of anti-Tat antibodies on HIV infection.

The Tat protein of the invention can be used to raise anti-wild-type Tat antibodies in mammalian systems susceptible to AIDS without otherwise compromising the health of the mammal. Such antibodies can be used to further study the immune response to HIV, in HIV assays, as well as to treat HIV infection.

The Tat protein of the invention can be used to produced monoclonal antibodies by methods well known in the art directed against specific epitopes of the protein. These antibodies could be used for passive Immunotherapy of HIV-infected individual in combination with chemotherapy and or therapeutic vaccination.

The said monoclonal antibodies can be used in ELISA assays. They are particularly useful as a prognostic tool to detect Tat antigenemia in course of HIV-infection inasmuch as the serum concentration of Tat is correlated with the number of HIV-infected cells.

Furthermore, the Tat protein of the invention can be used in ELISA assays to detect anti-Tat antibodies present in the serum of treated or non treated HIV-infected patients since high level of anti-Tat antibodies correlates with non progression to disease as demonstrated by Zagury et al. (J. of Human Virology, 1998, 1, 282-292). In such a case the protein of the invention is coated on an ELISA plate, contacted with serial dilutions of the patient serum to be tested, and then contacted with a enzyme-linked anti-human antibody. The anti-human antibody/anti-Tat anti-body complex thus formed is then detected by calorimetric detection. The Tat protein of the invention can be advantageously us as a negative control in any assay aiming to evaluate the transactivating and/or immunosuppressive activity of a Tat protein.

The tat/rev/nef expression vector of the invention can be used in ELISPOT assays to measure cellular responses in seropositive individuals as well as vaccinated individuals immunized with a different vector. Indeed, Tat and Rev responses have been shown to correlate with long-term non-progression. Carel A. Van Baalen et al., J. of General. Virology 78, 1913-1918 (1997).

Another use of the mutated Tat protein of the invention is as a research tool to study the immune response to HIV Tat during HIV infection. The mutated Tat protein of the invention enables scientists to observe the immune response to Tat in a model in vivo system without the presence of the complicating molecular processes of HIV gene expression and Tat induced immunosuppression.

The following examples further illustrate the invention and are not intended, nor should they be construed as limiting the invention in any manner. Those skilled in the art will appreciate that variations of the Examples provided below can be made in accordance with the teachings herein and knowledge common to those skilled in the art without varying from the scope or spirit of the present invention.

### EXAMPLES

### Example 1

### Construction of Plasmid pET8cTat7C/S

The construction of this clone involved two steps:

I the directed mutagenesis of the WT-tat gene to obtain the triple-mutant clone: Cys 30, Cys 31, Cys34→Ser 30, Ser 31, Ser 34.

II the directed mutagenesis of the tat-triple-mutant gene to obtain the pET8cTat7C/S plasmid.

Mutagenesis and Cloning of the Triple Mutant of Tat

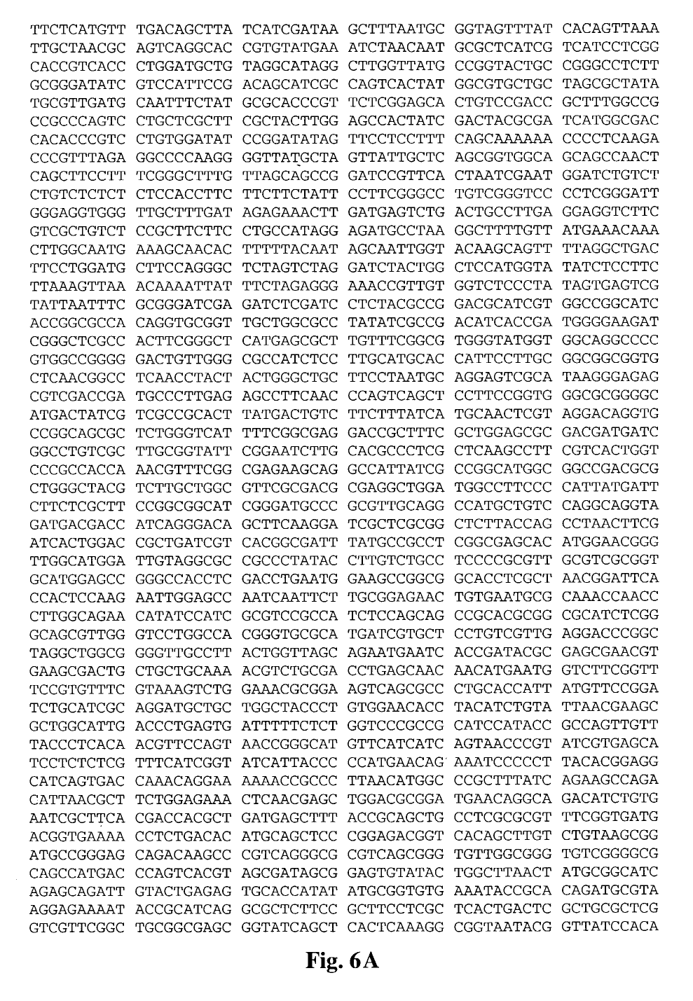

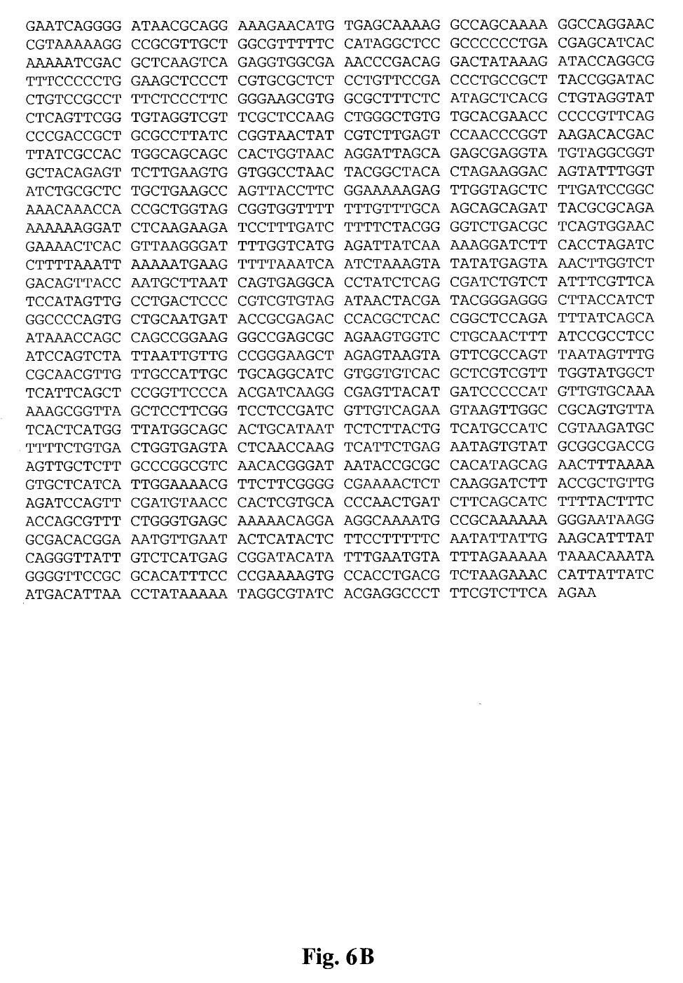

We used the recombinant PCR technique to mutate the WT-tat IIIB gene. The template was the clone pET8cTat (containing Seq. ID. No. 2). The map of this plasmid is given in FIG. 5 and its entire DNA sequence is given in FIG. 6. The recombinant PCR technique requires two PCR steps.

In the first step, two PCR reactions lead to the amplification and the mutagenesis of two overlapping fragments: the “5′ fragment” and the “3′ fragment” of the tat gene.

In the second round, the two overlapping fragments are mixed together along with 5′ and 3′ primers to amplify the whole mutated tat gene. In the strategy outlined below, nucleotide positions in the PCR primers corresponding to targeted alterations are underlined.

The protocol used was:

1. First round of PCR: amplification and mutagenesis of the 5′ fragment using the following primers: PBAMU(5′-CGCGGATCCATGGAGCCAGTAGATCCTA-3′) (SEQ ID NO.: 3) and R8 (5′ GTTATGAAACAAACTTGG\ddot{G}MTGAAAG\ddot{G}AA\ddot{G}ACTTT-3′) (SEQ ID NO.: 4) and amplification and mutagenesis of the 3′ fragment using the following primers: PHINDR (5′ CCCCAAGCTTCACTMTCGMTGGATCT-3′) (SEQ ID NO.: 5) and U8 (5′-AAAGT\ddot{C}TT\ddot{C}CTTTCATT\ddot{C}CCAAGTTTGTTTCATMC-3′) (SEQ ID NO.: 6)

2. Purification of these two PCR products using a preparative 2.5% agarose gel and a Qiagen gel extraction Kit (Qiagen, Valencia, Calif.)

3. Second round of PCR: amplification of the whole mutated gene using both fragments from the first round of PCR and the two primers: PBAMU and PHINDR

4. Purification of the 327 bp triple-mutated Tat-gene using a preparative 2.5% agarose gel and a Qiagen gel extraction Kit (Qiagen, Valencia)

5. Digestion of these DNA fragment by Hind III and Bam HI and purification of the fragment using a Qiagen PCR Extraction Kit.

6. Ligation of the digested fragment into pET8c vector previously digested with Bam HI and Hind III, transformation of XL 10 competent bacteria (Invitrogen, Carlsbad) with the ligation mix and mini-preparation of plasmids from cultures grown from the transformants using Qiagen Mini-Prep kit (Qiagen, Valencia)

7. Restriction analysis of the clones obtained and DNA sequencing.

Mutagenesis and Cloning of the 7-Serine Mutant of tat

The recombinant PCR technique was used with the triple mutant clone (obtained in the previous step) as template.

However, we needed 3 PCR steps to successfully amplify and mutate the whole gene. We were unsuccessful initially in trying to perform the recombinant PCR step with the initial length of overlap, so we extended the 5′ PCR products to increase the length of overlap between the two PCR products to be recombined in the final step. By combining the extended mutated 5′ fragment with the 3′ fragment in a third round of PCR using the 5′ and 3′ terminal primers, we were able to generate the full length 7C/S fragment.

The protocol used was:

1. First round of PCR: amplification and mutagenesis of the 5′ fragment using the following primers PBAMU (5′ CGCGGATCCATGGAGCCAGTAGATCCTA-3′) (SEQ ID NO.: 3) and R9 (5′ AAAG\ddot{G}AA\ddot{G}ACTTTTTA\ddot{G}AATAG\ddot{G}AATTGGTA\ddot{G}AAGCAGTTTT-3′) (SEQ ID NO.: 7) and amplification and mutagenesis of the 3′ fragment using the following primers PHINDR (5′ CCCCMG-CTTCACTAATCGMTGGATCT-3′ (SEQ ID NO.: 5) and U10 (5′-TAAAAAGT\ddot{C}TT\ddot{C}CTTTCATT\ddot{C}CCAAGTTT-\ddot{C}TTTCATAACAAA-3′) (SEQ ID NO.: 8)

2. Purification of these two PCR products using a preparative 2.5% agarose gel and a Qiagen gel extraction Kit (Qiagen, Valencia)

3. These two fragments failed to generate the full length Tat fragment in a secondary PCR reaction. Therefore we extended the 5′ fragment to increase the region of over-lap. This step # 3 enabled the extension of the 5′ fragment by PCR using the primers PBAMU(5′-CGCGGATCCATGGAGCCAGTAGATCCTA-3′) (SEQ ID NO.: 3) and R11(5′-GAAA\ddot{G}AAACTTG-G\ddot{G}AATGAAAG\ddot{G}AA\ddot{G}ACTTTTTA\ddot{G}AATAG\ddot{G}-3′) (SEQ ID NO.: 9)

4. Purification of this extended fragment using a preparative 2.5% agarose gel and a Qiagen gel extraction Kit (Qiagen, Valencia)

5. Third round of PCR amplification of the whole mutated gene using both fragments from the first round (fragments 3′, step # 1) and second round of PCR (extended fragment 5′, step #3) and the two primers: PBAMU and PHINDR

6. Purification of the 327 bp 7-ser-mutant-Tat-gene using a preparative 2.5% agarose gel and a Qiagen gel extraction Kit (Qiagen, Valencia)

7. Digestion of these DNA fragment by Hind III and Bam HI and purification of the fragment using a Qiagen PCR Extraction Kit.

8. Ligation of the digested fragment into pET8c previously digested with Bam HI and Hind III, transformation of XL 10 competent bacteria (Invitrogen, Carlsbad) with the ligation mix and mini-preparation of plasmids using Qiagen Mini-Prep kit (Qiagen, Valencia)

9. Restriction analysis of the clones obtained and DNA sequencing for confirmation of the desired construct.

### Example 2

### Construction of Plasmid pM1815

The Tat7C/S gene was inserted in the plasmid pET8cTat7C/S of example 1 between the BamH1 and HindIII sites. Since the ATG start site was immediately downstream of the Bam HI site (ggatccATGg) in the pET8cTat7C/S, this created an NcoI site (CCATGG) at the translation initiation codon. This NcoI site permitted direct insertion without modification of the reading frame in the pM1800 plasmid. This gene was therefore reinserted in this plasmid between the 5′NcoI and 3′HindIII sites.

The plasmid pM1800 is constructed starting from pET28 (Novagen). pET28c was amplified by PCR using two primers flanking either side of the region corresponding to the origin f1. The product thus amplified corresponds comprises the whole sequence of the vector with the exception of the region comprising origin f1. The two restriction sites Asc I and Pac I are introduced via the two primers used in the PCR reaction. In parallel the cer fragment is amplified using two primers which lead to a cer fragment inserted between Asc I and Pac I sites. The vector and the cer fragment thus amplified are digested by the Asc I and Pac I enzymes and then ligated together.

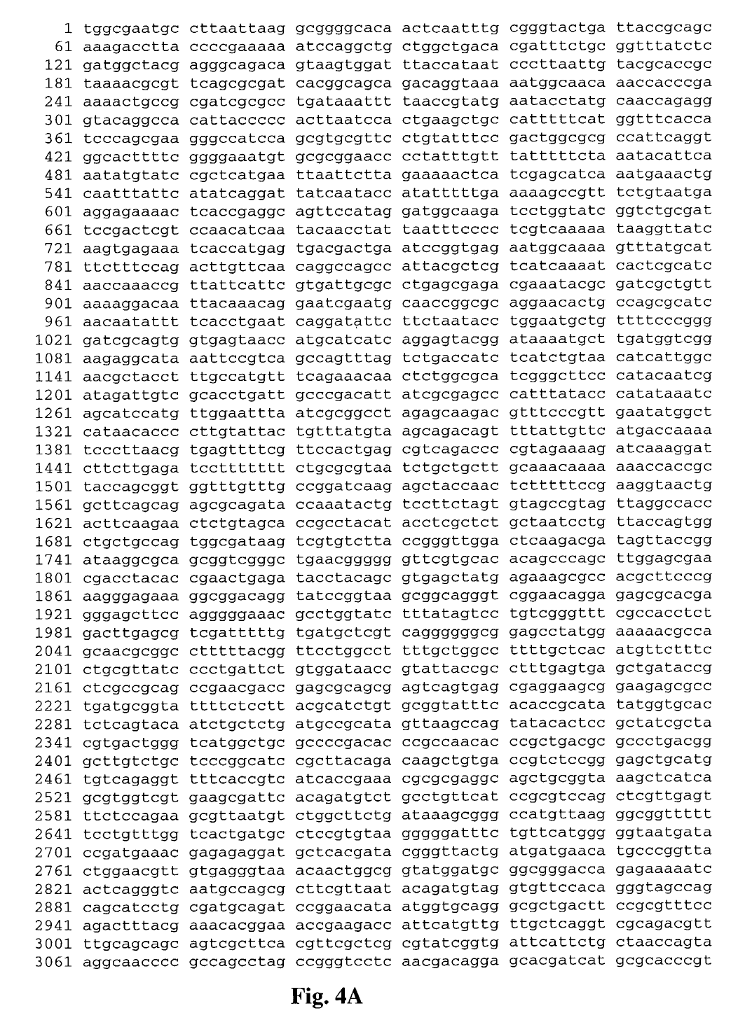

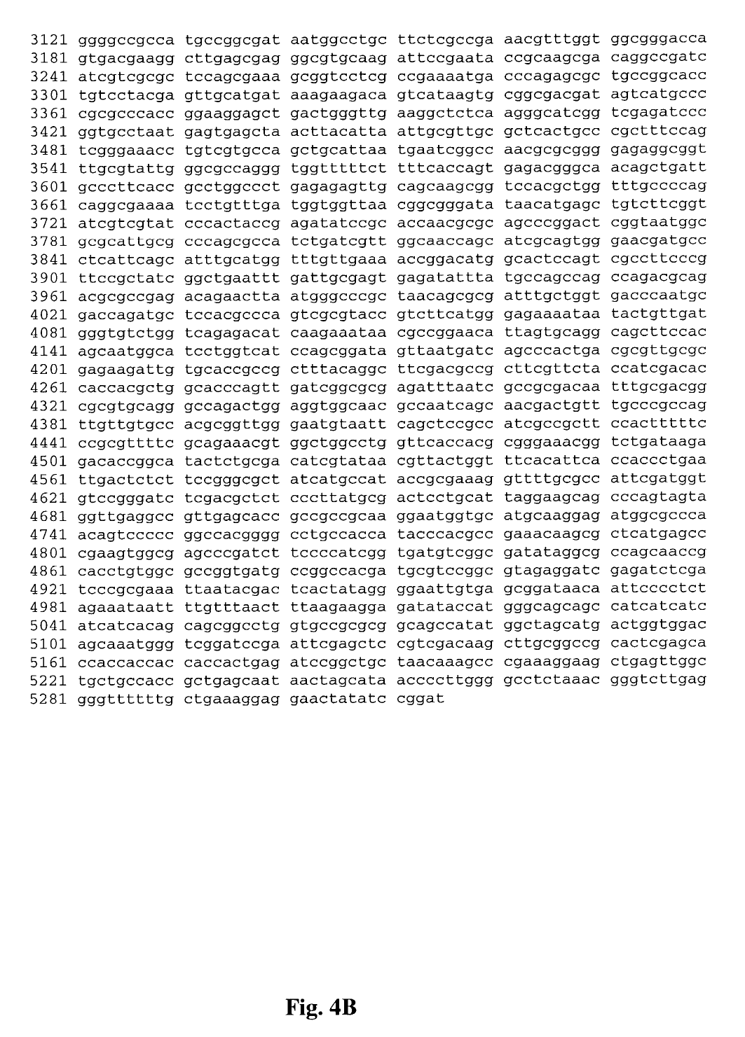

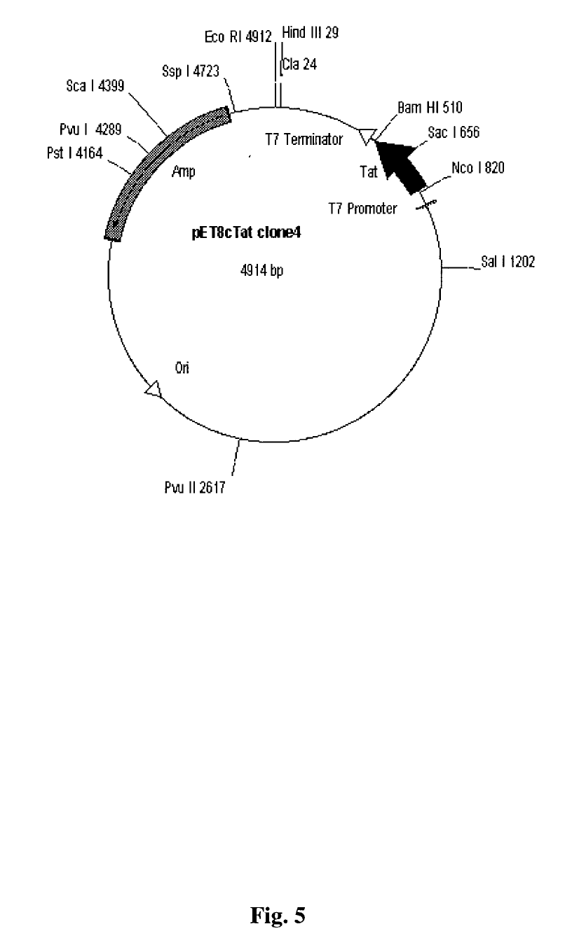

The vector pM1800 thus obtained comprises an expression cassette under the control of the bacteriophage T7 promoter, a polylinker for cloning the genes of interest downstream from the promoter, a transcription terminator also derived from bacteriophage T7, the cer fragment downstream the polylinker and a kanamycin resistance gene. The DNA sequence of plasmid pM1800 is given in attached FIG. 4.

The XL 1-Blue strain (Stratagene, La Jolla, Calif.) was transformed with pET8cTat7C/S. Two clones were transplanted and the ADN of the plasmid was extracted and digested with NcoI and HindIII (GIBCO-BRL) restriction enzymes in buffers suggested by the manufacturer. The Tat7C/S DNA sequence (approximately 300 bp) was then isolated on 2% agarose gel by electroelution.

At the same time, the pM1800 plasmid was also digested by NcoI and Hind III and isolated on 1% agarose gel by electroelution.

The digested Tat fragment and pM1800 plasmid were then subjected to ligation with the T4 ligase (GIBCO-BRL), under the conditions described by the manufacturer. The ligation product was used to transform the E. coli DH10B strain by electroporation, with the clones being selected in the presence of kanamycin.

The plasmid thus produced containing the Tat7C/S DNA sequence is named pM1815.

### Example 3

### Fermentation, Bacterial Cell Lysis and Protein Purification

A seed vial of pM1815 is used to inoculate, a pre-culture of E. coli BL21 (XDE3) (in Erlenmeyer flask containing the LB2X medium. After 15 h to 18 h agitation at 37° C., the whole content of the flask is added to 20 L of GluSKYE4 medium (yeast extract, salts and glucose) in a 30L B. Braun fermenter. When the initial growth phase reaches cell density up to A₆₀₀ of 30±5, the synthesis of the Tat protein IIIB 7C/S is induced by the addition of an inducer (IPTG 1 mM final). The culture is still maintained for 3 hours under agitation at 37° C. and then the medium is chilled down to 10° C. before cell harvesting. The cells are collected by centrifugation and stored at <−35° C.

| | |
|---|---|
| Thawing of bacterial paste (15 g) | Cellular paste thawed for 1 night at |
| | 5 ± 3° C. |
| ↓ | |
| Suspension and homogenization | In a buffer of 50 mM Tris-HCl, 0.2 |
| | M NaCl, benzonase* 5 Ul/ml, pH |
| | 8.0 in an ice bath at 5 to 10° C. |
| | using an Ultraturax |
| | * Addition of benzoates extempor- |
| | aneously |
| ↓ | |
| Cell lysis | High-pressure cracking using a |
| | Panda microfluidizer at 16000 |
| | psi or 1100 bars |
| | Centrifugation at 20000 g at |
| | 5 ± 3° C., for 2 hours |
| | Removal of supernatant and its |
| | clarification by filtration |
| | (0.8/0.2 μm) |
| ↓ | |
| Ammonium sulfate precipitation | Addition of ammonium sulfate to |
| | 1.5 M concentration |
| | Magnetic agitation, for 1 hour at |
| | room temperature |
| | 1 hour of rest |
| | Centrifugation at 10000 g at |
| | 20 ± 3° C. |
| | Re-suspension of ammonium |
| | sulfate (AS) precipitate |
| | in a 50 mM Tris-HCl, 8 M |
| | Urea, 50 mM NaCl, pH 8.0 |
| | buffer = AS solution. |
| ↓ | |
| SP Sepharose FF Chromatography | column Equilibrated in a buffer |
| volume 20 ml | of 50 mM Tris-HCl, 50 mM NaCl, |
| 1.5 cm | 8 M urea |
| Flow rate 2 ml/min | Injection of the filtrated AS |
| | solution followed by rins- |
| | ing with pH balance-restoring |
| | buffer solution |
| | Removal of the flow through |
| | Elution with increasing ionic |
| | strength |
| | 50 mM Tris-HCl, 0.3 M NaCl, |
| | 8 M urea, pH 8.0 |
| | 50 mM Tris-HCl, 0.6 M NaCl, |
| | 8 M urea, pH 8.0 |
| | 50 mM Tris-HCl, 1.5 M NaCl, |
| | 8 M urea, pH 8.0 |
| | Tat7C/S eluted in the NaCl 0.6 M |
| | eluate |

The purified Tat protein is stored at −20° C. The buffer of the Tat protein thus purified is preferably replaced with an urea-free buffer such as 50 mM Tris-HCI pH 7.5. Furthermore, the Tat protein needs to be sterilized before injection. This step can be easily done by sterilizing filtration on 0.2 μm membrane. The Tat IIIB 7C/S thus isolated is greater than 95% pure, as determined by densitometric analysis on a blue coomasie-stained SDS-PAGE gel. Furthermore, the protein thus purified is substantially exempt of any multimeric forms. Indeed, and contrary to the preparation of the Tat protein of the prior art, the protein thus produced is a monomeric protein containing less than 1% of multimeric Tat forms.

Furthermore, the protein of the invention can be purified at a pH near neutrality without forming aggregates. Furthermore, it appears that the expression level of the protein of the invention is higher than the expression level of the corresponding wild-type protein. Indeed Wild-type Tat represents 5% of the total soluble proteins whereas Tat7C/S represents at least 15% of the total soluble proteins.

### Example 4

### Neutralization and Neutralization Assays

Transactivation Assay

The transactivation assay was developed from G. Tosi et al., Eur. J. Immunol. 30, 1120-1126 (2000) and M. Rusnati et al. J. of Biological Chemistry 272, 11313-11320 (1997), allowing the biological activity of the Tat molecule to be determined in vitro. Stably transfected HeLa-3T1 cells are carrying a plasmid with the LTR sequences of the HIV virus. These LTR sequences function as a promoter for the gene of the chloramphenicol acetyl transferase (CAT) which is a reporter. The addition of Tat to the culture medium causes the synthesis of CAT, which can be measured with a commercial ELISA test (Boehringer). The results were standardized in relation to the cellular protein concentration.

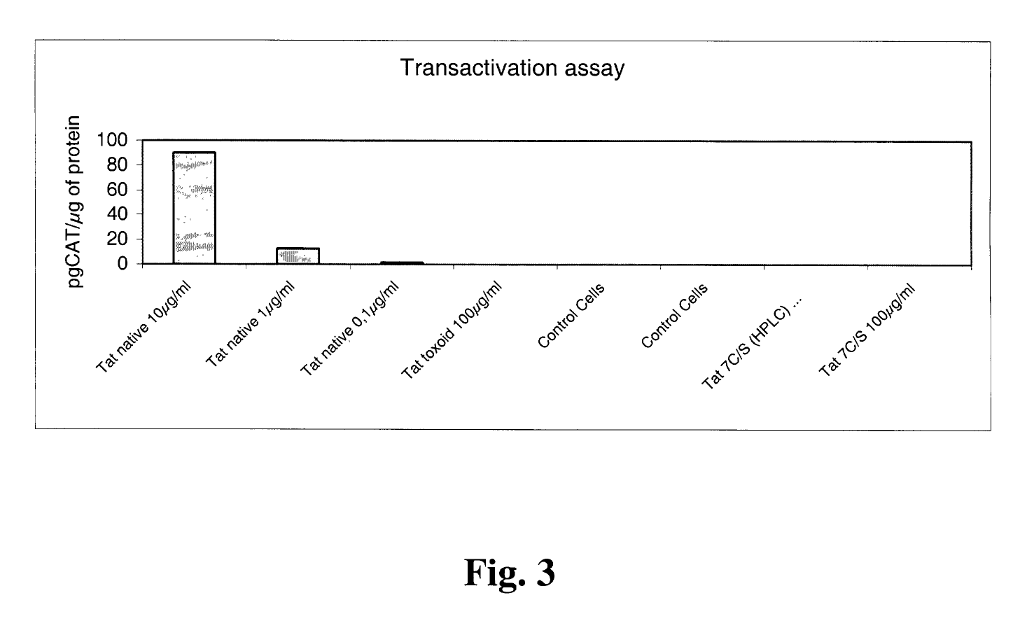

FIG. 3 is showing the transactivating activity of the native Tat, Tat toxoid and Tat7C/S.

Neutralization Assay

The incubation of serial dilutions of sera with 40 ng/ml of purified native Tat prior to transactivation assay, allows to check for neutralization of transactivation activity by comparison with adequate controls.

Neutralizing titers are expressed as reverse of the last dilution able to reduce 90% (1log) of the transactivation signal.

The following table shows specific antibody titer and neutralizing titer:

TABLE 1

| | | Specific antibody |
|---|---|---|
| Sample tested | Neutralizing titer | titer (log) |
| Cob #075-5 (Tat Toxoid) | <or = 5 | 3.79 |
| Cob #075-33 (Tat7C/S) | 5 | 3.45 |
| Cob#074 (native Tat) | 5 | 3.2 |
| Cob#045 (positive control) (hy- | 800 | 6.3 |
| per-immune anti-Tat serum | | |
| (CFA)) | | |

These results clearly indicate that Tat7C/S induce antibodies which neutralize Tat transactivation activity. The neutralizing titer is equivalent to titer obtained with Tat toxoid.

Moreover, this experiment confirm that the neutralization test is very sensitive since a neutralizing activity is measured even with low titer sera.

### Example 5

### Immunosuppression Assay

The immunosuppressive activity of Tat was measured in vitro by a lymphoproliferation assay. Lymphoproliferation was measured by tritiated thymidine incorporation (³H-thymidine) in peripheral blood mononuclear cells (PBMCs) after stimulation by a recall antigen (previously described in Zagury et al., Proc. Nati. Acad. Sci. USA. 1998; 95:3851-6).

This assay consisted of isolating, on a ficoll gradient, PBMCs from the peripheral blood of a healthy subject and cultivating them in a microwell in the presence of recall antigen and declining doses of Tat protein in an HL1 culture medium supplemented with 5×10⁻⁵M B-mercaptoethanol and 10% AB serum. Each dose of Tat was tested in triplicate. 18 hours before the cessation of the culture, 0.5 mCi of tritiated thymidine was added to each microwell. The cells were then washed and the incorporated radioactivity was measured with a fluid scintillation counter. The results were measured in cpm.

The goal of this test was to characterize the immunosuppressive properties of a genetic mutant of Tat. The PBMCs were incubated with 5 pg of native Tat IIIB, detoxified or Tat7C/S), stimulated by the antigens PPD/TT (PPD at 1000 units/ml and TT at 1000 Lf/ml) over a period of 5 days.

Detoxified Tat is produced by inactivation of Tat IIIB by an alkylation reaction of Tat IIIB (Seq. ID. No. 1) using iodoacetamide in the following conditions: added micromoles of iodoacetamide=200×number of micromoles of Tat+number of micromoles of DTT.

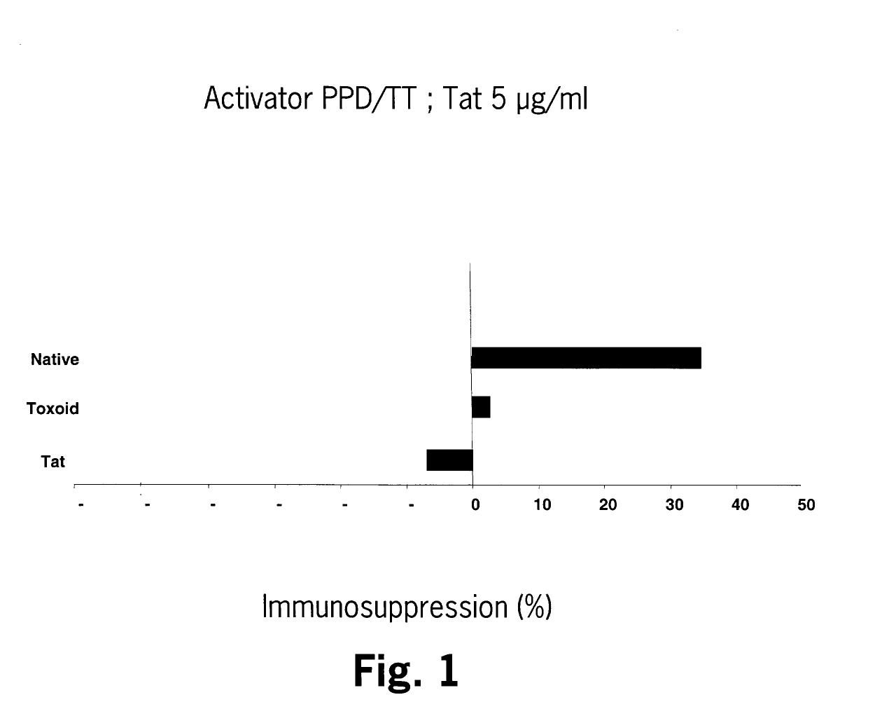

The results are presented in FIG. 1 as % of immunosuppression, calculated as follows:

$$\% immunosuppression = \frac{( cpm in cells not treated with Tat ) - ( cpm in cells treated with Tat ) }{100}$$

The data represent 3 experiments performed independently on 3 different donors. The results show that under conditions where native Tat inhibits the proliferation of PBMCs by 40%, the mutant of Tat7C/S shows no immunosuppressive activity.

### Example 6

### Immunogenicity of the Mutant Tat_{IIB} 7C/S in the Guinea Pig

Five female guinea pigs (Dunkin-Hartley albinos) were injected two times, at two week intervals, intramuscularly (in the quadriceps) with 50 μg of the TatIIIB 7C/S. A control group of five guinea pigs received, in a similar manner, 50 μg of chemically detoxified TatIIIB protein (termed “TatIIIB toxoid” prepared according to the process described in example 5).

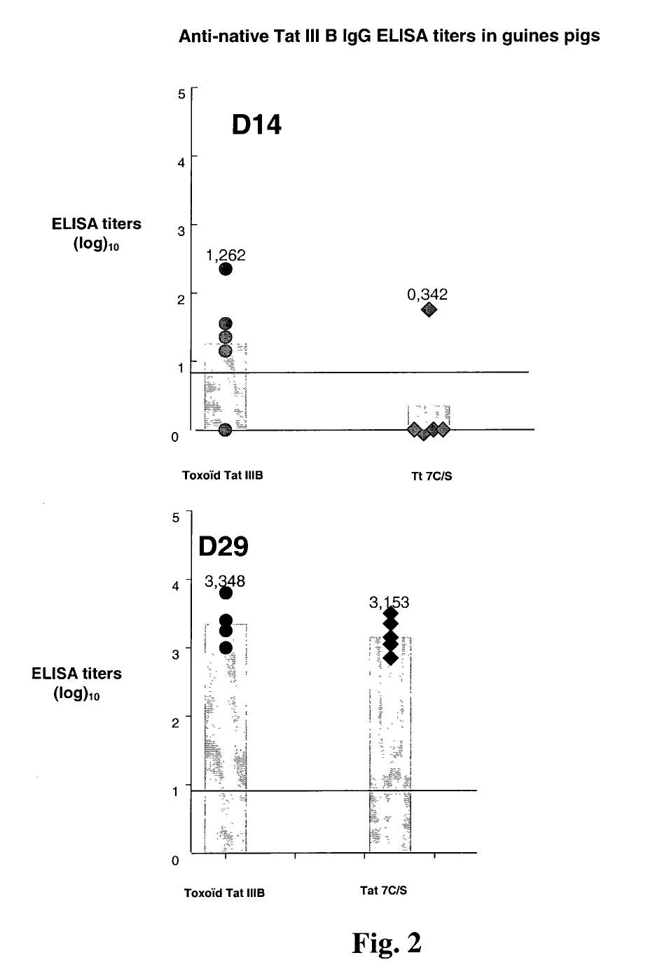

The antibody level induced against the native Tat_{IIIB} protein were evaluated by ELISA before and after each immunizations (Days 1, 14, and 29, respectively). The results are displays in FIG. 2.

The IgG antibody titers (expressed in log₁₀) are represented in the table 2. The antibody titers of the samples were calculated by linear regression of a standard an anti-Tat_{IIIB} hyperimmune serum from guinea pig. The titer of this standard serum was first set as the reciprocal of its dilution, giving an optical density at 450-650 nm of 1.0 (average titer calculated at the end of several independent titrations). Limit of detection set at 0.7 log₁₀.

The Tat_{IIIB} 7C/S was shown to be capable of inducing specific antibodies against the native Tat_{IIIB} protein in this guinea pig model, with the levels induced after 2 immunizations being very close to those evoked by the Tat_{IIIB} toxoid protein.

TABLE 2

| | | Native anti-Tat_{IIIB} IgG antibody titers | | |
|---|---|---|---|---|
| | | (log₁₀) | | |
| Immunogen | Guinea pig # | Day 1 | Day 14 | Day 29 |
| Tat_{IIIB} 7C/S | 1 | 0 | 0.000 | 3.327 |
| | 2 | 0 | 0.000 | 3.112 |
| | 3 | 0 | 1.711 | 3.458 |
| | 4 | 0 | 0.000 | 2.834 |
| | 5 | 0 | 0.000 | 3.034 |
| | mean | 0 | 0.342 | 3.153 |
| | std deviation | 0 | 0.765 | 0.245 |
| Toxoid Tat_{IIIB} | 6 | 0 | 0.000 | 3.243 |
| | 7 | 0 | 1.327 | 2.967 |
| | 8 | 0 | 1.522 | 3.384 |
| | 9 | 0 | 1.138 | Dead |
| | 10 | 0 | 2.321 | 3.796 |
| | mean | 0 | 1.262 | 3.348 |
| | std deviation | 0 | 0.837 | 0.346 |

We claim:

1. A Tat protein comprising a mutated cysteine-rich domain wherein all the cysteine residues of the cysteine-rich domain have been replaced independently with another amino acid.

2. The Tat protein according to claim 1, wherein each cysteine residue of the cysteine-rich domain is a conservative substitution.

3. The Tat protein according to claim 1, wherein each cysteine residue of the cysteine-rich domain is a serine.

4. A nucleic acid encoding the Tat protein according to any one of claims 1 to 3.

5. An expression vector comprising a nucleic acid according to claim 4.

6. The expression vector of claim 5 further comprising a DNA sequence encoding Nef and Rev proteins.

7. The expression vector of claim 6 wherein the DNA sequence encoding the Rev protein is inserted anywhere into the Nef DNA sequence encoding amino acids 150-179 of the Nef protein.

8. A composition comprising the Tat protein according to any one of claims 1 to 3, carrier and optionally and adjuvant.

9. A composition comprising the expression vector according to any one of claims 5 to 7, a carrier and optionally an adjuvant.

10. The composition of claim 8 or 9 comprising at least one Th1 adjuvant.

11. A method of eliciting a humoral and cellular immune response in a mammal comprising administering a composition according to any one of claims 8 to 10 to the mammal.

12. The method according to claim 11 wherein the composition of claim 8 and the composition of claim 9 are administered simultaneously or sequentially.
